(19) United States
(12) Patent Application Publication
(10) Pub. No.: US 2026/0294271 A1
(43) Pub. Date: Oct. 1, 2026

(54) EQUINE ALLERGIC AIRWAY DIAGNOSIS AND ENVIRONMENTAL AIR AND ALLERGEN ASSESSMENT

(71) Applicant: Texas Tech University System, Lubbock, TX (US)

(72) Inventors: Laszlo Hunyadi, Amarillo, TX (US); Emily Sundman, Amarillo, TX (US)

(21) Appl. No.: 19/481,663

(22) PCT Filed: May 2, 2024
(86) PCT No.: PCT/US2024/027530

§ 371 Date: Nov. 5, 2025

Related U.S. Application Data
(60) Provisional application No. 63/464,254, filed on May 5, 2023.

(51) Int. Cl. A61B 5/08; A61B 5/00; A61B 5/097

(52) U.S. Cl. CPC A61B 5/082; A61B 5/097; A61B 5/4848; A61B 2503/40; A61B 2560/0242

(57) ABSTRACT

A monitoring system for equine allergic airway diagnosis and environmental and air allergen assessment comprises a collection muzzle, a sensor in fluidic connection with the collection muzzle, an environmental sensor disposed in an environment proximate to the collection muzzle, and a chemical sensor disposed in the environment proximate to the collection muzzle.

### CROSS REFERENCE TO RELATED PATENT APPLICATIONS

This application claims the priority and benefit under 35 U.S.C. § 119(e) of U.S. Provisional Patent Application Ser. No. 63/464,254 filed May 5, 2023, entitled “EQUINE ALLERGIC AIRWAY DIAGNOSIS AND ENVIRONMENTAL AIR AND ALLERGEN ASSESSMENT.” U.S. Provisional Patent Application Ser. No. 63/464,254 is herein incorporated by reference in its entirety.

### TECHNICAL FIELD

Embodiments are generally related to the field of veterinary diagnostic tools and methods. Embodiments are also related to the field of allergen diagnosis. Embodiments are further related to equine asthma diagnosis. Embodiments are also related to equine diagnostic masks. Embodiments are also related to environmental monitoring of animal environments.

### BACKGROUND

Equine asthma is a debilitating disease negatively impacting the health and utility of horses. Clinical presentations of the disease vary according to the use of the horse, but asthma often remains underdiagnosed until advanced clinical signs become observable (e.g., severe asthma or recurrent airway obstruction such as heaves).

Identifying causes of exercise intolerance in the equine athlete has led to a growing interest in identifying earlier, and milder, forms of asthma. Severe clinical signs (bronchospasm, tachypnea, coughing) are more widely recognized with asthma and reflect the pathophysiology of ongoing airway inflammation; including recruitment of immune cells, bronchoconstriction, and mucus hypersecretion. Failure to control the initial inflammatory cycles may lead to severe disease in some horses which can be career or performance limiting.

The pathophysiology of equine asthma mirrors, in some respects, the pathophysiology of asthma in humans. Broadly, asthma is a non-infectious inflammatory condition of the respiratory system. Environmental exposure to irritants (e.g., allergens, pollutants, toxins, small particulates) initiates the inflammatory cycle that diminishes the normal pulmonary micro-environment, leading to an increased risk for secondary respiratory infections. Much as in human medicine, environmental modification to decrease exposure to the allergens and irritants is one approach to mitigating asthma symptoms. However, in practice this can be challenging, or even impossible to implement. Horses live in environments where some allergens cannot be avoided (e.g., amongst grasses, weeds, and trees, in barns and arenas, etc.) despite the best efforts of the care giver.

Asthma can be either neutrophilic, eosinophilic, mastocytic, or a combination of cell-types based on the degree of elevation of cell-type cytology. Lung function testing, analogous to human diagnostic testing, requires referral to a select few tertiary equine hospitals with the equipment necessary to complete the evaluation. In addition, commercially available equine lung function testing is not available, and therefore test results are not applicable across locations.

Traditionally, a horse within an environment that has poor air quality will demonstrate clinical signs of asthma (increased respiratory rate, increased heart rate, cough, phlegm production and exercise intolerance). These horses undergo a bronchoalveolar lavage (BAL). The fluid aliquot obtained from the procedure is spun down, concentrated, and sent to a diagnostic lab for cytological reading by a board certified clinical pathologist. The presence of inflammatory cells as well as the type of inflammatory cell that predominates can be identified. This cytological interpretation is then used to determine the type of asthma the horse has (neutrophilic vs eosinophilic or mastocytic or a combination of either of these cell types).

Diagnosis of equine asthma historically has relied on cytological evaluation of bronchoalveolar lavage (BAL) fluid. The BAL procedure, whether completed via a BAL catheter or under endoscopic guidance, requires technical expertise. There is very likely a significant underdiagnosis of asthma in horses. This is due in part to the technical challenges of completing a BAL procedure and the subjective evaluation and interpretation of BAL cytology by clinical pathologists.

Given the value of horses and the increasing need to correctly diagnose asthma, there is a need for systems and methods for identifying equine asthma as disclosed herein.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide a diagnostic system.

It is another aspect of the disclosed embodiments to provide a diagnostic method.

It is another aspect of the disclosed embodiments to provide methods and systems for monitoring environmental air and allergens.

It is another aspect of the disclosed embodiments to provide methods, systems, and apparatuses for identifying equine asthma.

It is another aspect of the disclosed embodiments to provide methods, systems, and apparatuses for equine allergic airway diagnosis and environmental air and allergen assessment.

It will be appreciated that the methods and systems can be achieved according to the embodiments disclosed herein. For example, in an embodiment, a monitoring system comprises a collection muzzle, a sensor in fluidic connection with the collection muzzle, an environmental sensor disposed in an environment proximate to the collection muzzle, and a chemical sensor disposed in the environment proximate to the collection muzzle. In an embodiment, the sensor comprises a nitric oxide monitor. In an embodiment, the environmental sensor comprises a particle monitor. In an embodiment, the particle monitor is configured to detect particles of 10 microns or less. In an embodiment, the chemical sensor further comprises a sensor configured to measure at least one of ammonia, carbon monoxide, methane, and/or hydrogen sulfide. In an embodiment, the collection muzzle further comprises: a collection chamber and a sealing gasket formed on a rim of the collection chamber. In an embodiment, the collection muzzle further comprises an adaptor port configured to engage attachment tubing connecting the collection chamber to the sensor. In an embodiment, the collection muzzle is configured to cover the nose and mouth of a horse.

In an embodiment, the monitoring system further comprises a computer system comprising at least one processor and a storage device communicatively coupled to the at least one processor, the storage device storing instructions which, when executed by the at least one processor, cause the at least one processor to perform operations comprising: receiving nitric oxide indicia data from the sensor, determining a nitric oxide level from the nitric oxide indicia data; and determining if the nitric oxide level is above a threshold indicative of equine asthma. In an embodiment, the at least one processor performs operations comprising receiving environmental data from at least one of the environmental sensor, and the chemical sensor, analyzing the environmental data, and determining if the environmental data suggests environmental conditions causing equine asthma. In an embodiment, the at least one processor performs operations comprising receiving intradermal skin testing data, analyzing the intradermal skin testing data, and determining if the intradermal skin testing data suggests conditions causing equine asthma.

### BRIEF DESCRIPTION OF THE FIGURES

The accompanying figures, in which like reference numerals refer to identical or functionally similar elements throughout the separate views and which are incorporated in and form a part of the specification, further illustrate the embodiments and, together with the detailed description, serve to explain the embodiments disclosed herein.

FIG. 1 illustrates a block diagram of a monitoring system, in accordance with the disclosed embodiments;

FIG. 2A illustrates a collection muzzle associated with a monitoring system, in accordance with the disclosed embodiments;

FIG. 2B illustrates another view of a collection muzzle associated with a monitoring system, in accordance with the disclosed embodiments;

FIG. 2C illustrates another view of a collection muzzle with an integrated sensor associated with a monitoring system, in accordance with the disclosed embodiments;

FIG. 3A illustrates a method for equine allergic airway diagnosis, in accordance with the disclosed embodiments;

FIG. 3B illustrates a method for equine allergic airway diagnosis, and associated environmental air and allergen assessment using a monitoring system, in accordance with the disclosed embodiments;

FIG. 3C illustrates a method for monitoring the efficacy of a therapeutic and/or treatment regimen following diagnosis of equine asthma, in accordance with the disclosed embodiments;

FIG. 4 depicts a block diagram of a computer system which is implemented in accordance with the disclosed embodiments;

FIG. 5 depicts a graphical representation of a network of data-processing devices in which aspects of the present embodiments may be implemented; and

FIG. 6 depicts a computer software system for directing the operation of the data-processing system depicted in FIG. 4, in accordance with an example embodiment.

### DETAILED DESCRIPTION

Embodiments and aspects of the disclosed technology are presented herein. The particular embodiments and configurations discussed in the following non-limiting examples can be varied, and are provided to illustrate one or more embodiments, and are not intended to limit the scope thereof.

Reference to the accompanying drawings, in which illustrative embodiments are shown are provided herein. The embodiments disclosed can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

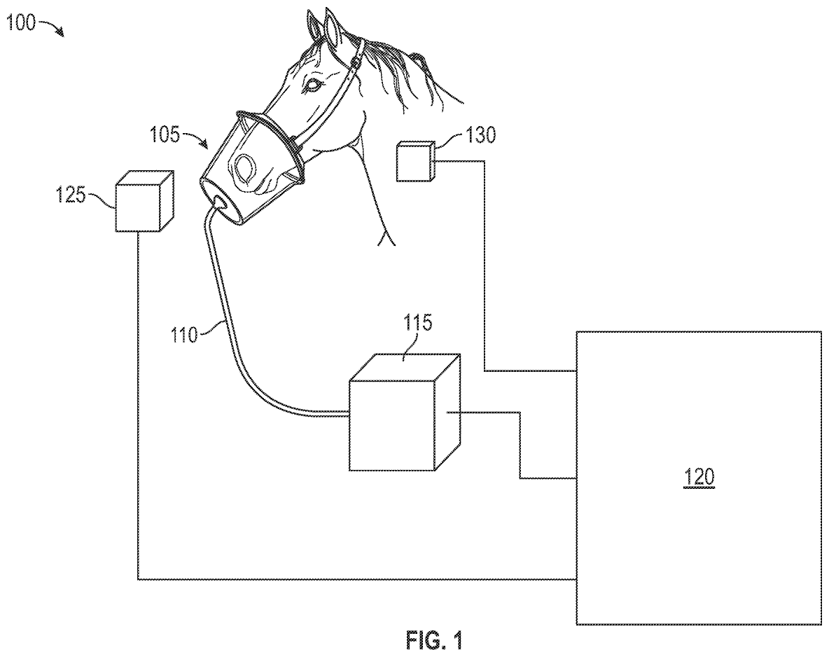

FIG. 1 illustrates a schematic block diagram of a monitoring system 100 in accordance with the disclosed embodiments. The monitoring system 100 generally comprises a collection muzzle 105 with attachment tubing 110 fluidically connecting the collection muzzle 105 to a sensor 115. The sensor 115 can be configured as a nitric oxide monitor. In other embodiments, the sensor 115 can be configured inside the collection muzzle 105 as further detailed herein.

Data from the sensor 115 can be provided to a processing system 120 which can input data from the sensor 115 and generate output data. In certain embodiments, the processing system 120 can comprise a computer system, tablet device, mobile phone, smart phone, or some combination thereof.

The monitoring system 100 can further include a second environmental sensor 125. In certain embodiments, the environmental sensor 125 can be mounted at or near the face level of the horse. For example, the environmental sensor 125 can be mounted to a wall or rail in a horse stall at roughly the height of the horse's face. In other embodiments, the environmental sensor can be mounted directly to the collection muzzle 105. Aspects of the environmental sensor 125 are further detailed herein.

In certain embodiments, the monitoring system 100 can also include a third chemical sensor 130. As with the environmental sensor 125, the chemical sensor 130 can be configured to be worn at or near the horse's face level. In certain embodiments, the chemical sensor 130 can be connected to, or internal to the environmental sensor 125 and/or the collection muzzle 105.

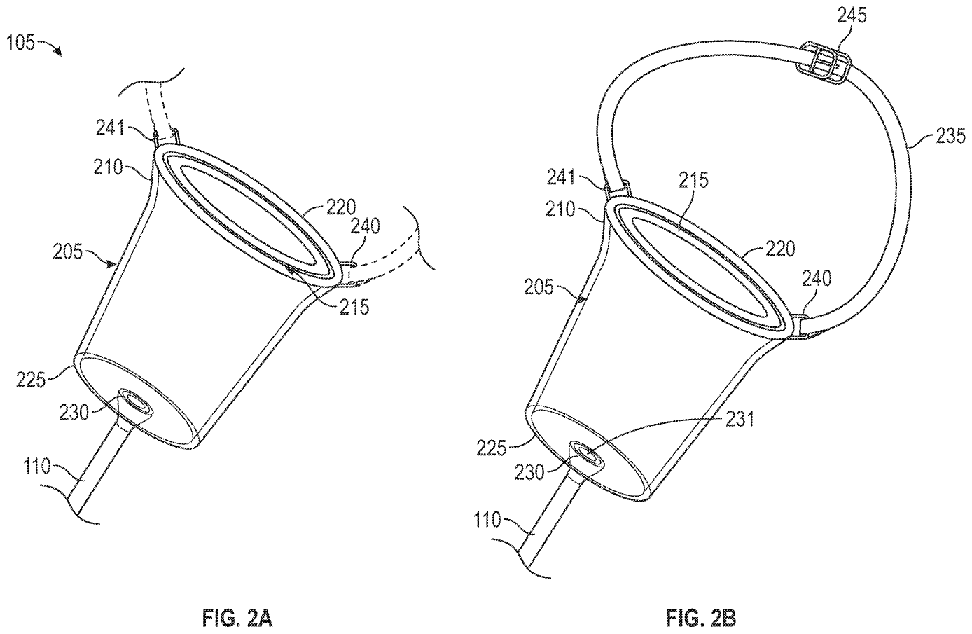

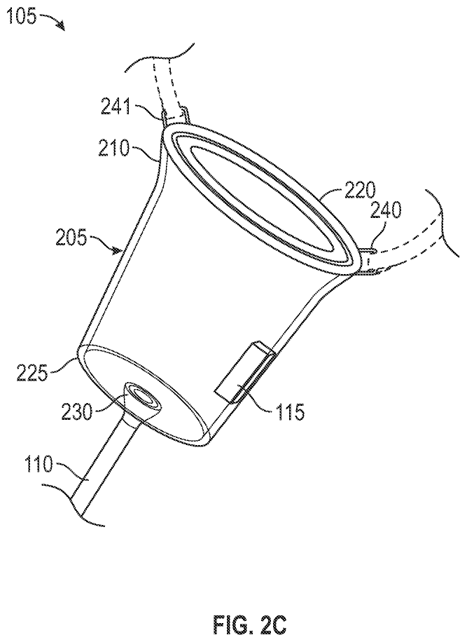

Aspects of the collection muzzle 105 are further detailed in FIG. 2A and FIG. 2B. The collection muzzle 105 comprises a collection chamber 205. The collection chamber 205 is configured to collect the exhalation of the horse. The collection chamber 205 is sized to have a sufficient diameter and depth to comfortably fit over the nose and mouth of a horse. The collection chamber 205 is fitted at one end 220 with a sealing gasket 210. The sealing gasket 210 can comprise a ring fitted to the rim 215 of the collection chamber 205. The sealing gasket 210 can be formed of a rubber or soft plastic material and is configured to form a comfortable seal around the horse's face, to prevent exhalation from escaping and to prevent the introduction of gas from outside the collection muzzle 105.

The collection muzzle 105 can further include a strap 235 as illustrated in FIG. 2B. The strap 235 is configured to attach on one end to a strap loop 240, and on the other end to strap loop 241. The strap loop 240 and strap loop 241 can be mounted on opposing edges of the rim 215 of the collection chamber 205. The strap 235 can be configured to include a size adjusting mechanism 245 such as a buckle, hook and loop fastener, or the like, so that the length of the strap can be adjusted to fit horses with different sized heads.

The other end 225 of the collection chamber 205 is configured with an adaptor port 230 configured to connect to attachment tubing 110. The adaptor port 230 can comprise tigon tubing which allows exhaled gas to flow to sensor 115. The adaptor port 230 can include a valve 231 to prevent backflow.

FIG. 2C illustrates additional aspects of the collection muzzle 105, when it is configured to include the sensor 115 inside the collection muzzle 105. As used herein, “inside” the collection muzzle 105 can mean mounted within, on, or integrated with, the collection muzzle 105. The collection chamber 205 is configured to collect the exhalation of the horse and the sensor 115 inside the collection muzzle 105 can sample such exhalation for the presence of nitric oxide. The collection chamber 205 is designed to have sufficient diameter and depth to comfortably fit over the nose and mouth of a horse, while accommodating the sensor 115. The sealing gasket 210 is configured to form a comfortable seal around the horse's face.

The sensor 115 can comprise a nitric oxide monitor configured to take serial measurements of nitric oxide exhaled by the horse. Traditionally, it is difficult to collect a sufficient nitric oxide sample from a horse to be of use for asthma diagnosis. It should be appreciated that the system 100 can be used for the detection of other conditions including, but not limited to, pneumonia and other such conditions. The disclosed embodiments, make use of a collection muzzle 105, which is fitted snugly around the horse's face, to allow a sufficient sample to be collected in the collection chamber 205, from the horse's exhalation in a sufficient concentration to be measured.

It should be appreciated that most of a horse's breath is captured during a nominally 3 second exhalation. In some embodiments, this can be completed pre-exercise and post-exercise so that measurements can be compared. An advantage of the current embodiments is that the horse need not hyperventilate to collect a sufficient sample.

The nitric oxide monitor 115 can then provide quantifiable measurements of nitric oxide through continuous readings. In certain embodiments, this could comprise readings taken at 2 minute intervals, but other sample timing is also possible.

The sensor reading from the sensor 115 can be provided to a processing system 120 for analysis and outputting measurement information. If readings from the sensor suggest a horse has elevated nitric oxide, as measured in the collected exhalation form the horse, then the horse is likely suffering from non-infectious pulmonary inflammation. If the measurements are normal, then the horse is likely ok. If the measurements are high, treatment is warranted which would usually coincide with nebulization (albuterol & budesonide) and or straight saline.

The system is primarily configured to evaluate the clinical utility of exhaled nitric oxide and to provide a stall-side diagnostic tool for eosinophilic and mastocytic asthma in horses. In accordance with the disclosed embodiments, if the system detects fractioned exhaled nitric oxide (FeNO) concentrations ≥20 ppb, there is a high likelihood the horse has asthma, and will be diagnosed with eosinophilic and/or mastocytic asthma if traditional diagnostic techniques were used.

For example, a normal level of nitric oxide should be less than 20 parts per billion. Anything above this threshold can be considered pathologic. Likewise, any level above 20 parts per billion would, with high probability, be indicative of allergic asthma.

The environmental monitor 125 can comprise a particle monitor, or other such environmental monitor, that takes in continuous measurements of particles within the air/environment/barn that are nominally 10 microns or less is size, and are able to be inhaled by horses and humans. In some embodiments, the environmental monitor 125 can comprise a DustTrack monitor but other environmental monitors can also be used.

The chemical sensor 130 can comprise, for example, a GM460 monitor. The chemical sensor 130 can be worn on the horse. The chemical sensor 130 is configured to take chemical measurements of molecules, including but not limited to, ammonia, carbon monoxide, methane, and/or hydrogen sulfide along with other chemicals as may be appropriate for the given environment. The chemical sensor 130 can collect serial measurements throughout the day, and can provide data to the processing system 120. The processing system 120 can be used to provide analysis on what the daily air quality is like in the horse's environment (e.g., barn and stall).

It should be appreciated that in certain embodiments, the environmental monitor 125 and chemical sensor 130 can be integrated into a single housing. For example, the environmental monitor 125, can be configured to measure environmental loads of particulates. The chemical sensor 130 can be housed inside the environmental monitor 125 and is configured to measure noxious chemical levels of NH₃, CO, SO₂ and the like. These monitors can be configured to vacuum air samples and process them in real time. The combined environmental sensor 125 and chemical sensor 130 can also be directly mounted to the halter on the horse in order to obtain noxious chemical levels near the horse's head (i.e., nostrils for inhalation).

In an embodiment, the system 100 further comprises a processing system 120 comprising at least one processor and a storage device communicatively coupled to the at least one processor, the storage device storing instructions which, when executed by the at least one processor, cause the at least one processor to perform operations comprising: receiving indicia data from at least one of the sensor, the environmental sensor, and the chemical sensor; analyzing the environmental data; and determining if the data is indicative of equine asthma.

The computer system can include an analysis module used to combine data from one or more of the monitors. This allows the system to assess each individual horse and/or the overall environment that the horse is experiencing. If the system identifies a horse with increased nitric oxide measurements, a veterinary exam can be scheduled. Asthmatic horses that are diagnosed with asthma can then be monitored with the nitric oxide monitor vs having a repeat BAL procedure. Successfully treated asthmatics will have normal nitric oxide measurements. Asthmatic horses with uncontrolled asthma will continue to have increased nitric oxide measurements. Data can also be used to drive development of treatment pathways or protocols based on the collected data.

In the disclosed embodiments, nitric oxide levels of less than 20 ppb are considered in a normal range. For particulates, fewer identifiable particulates is always better. However, identification of particulates that are less than 10 microns in size may be indicative of a higher risk environment since these smaller particulates can more readily enter the lungs. As a general principal the system can include a measurement on the air quality index (AQI) which can include ozone, particle pollution, sulfur dioxide, nitrogen dioxide, and carbon monoxide. An AQI above 100 is considered unhealthy for asthmatic horses. Even an AQI ranging from 50-100 can exacerbate symptoms of equine asthma.

As such, in certain embodiments, readings from the system 100 can be provided to the analysis module, which can generate an average AQI. This information can be used to determine if the horse's environment is creating risk of airway inflammation. If further testing indicates horses within a proximate environment (like a barn) have unusually high occurrences of asthma, the environmental data may suggest that dust mitigation, allergen control, etc. may be useful in reducing airway inflammation.

The data can also be used to evaluate ventilation in the nearby environment, as well as how the environment changes during cleaning times, rising times, and the like. For example, a horse with asthma is best removed from the barn during cleaning because this time usually correlates with high levels of particulate matter in the air. An asthmatic horse will be more sensitive to high levels of particulate matter in the air. By removing the horse from the barn during cleaning time, it may help decrease the chances of a flare up.

In an embodiment, data collected by the system 100 can be used for laboratory assessments. Exhaled nitric oxide can be assessed patient-side using an embodiment of the nitric oxide system to ensure sufficient exhalation pressure from the non-sedate horse. If the system indicates elevated nitric oxide levels, Bronchoalveolar lavage fluid can be cytocentrifuged and cytological evaluation of stained slides will be completed by a clinical pathologist. Based on the results horses can be diagnosed with asthma. Horses can be diagnosed as normal, mild to moderate asthma, or severe asthma. Asthma diagnosis will be further defined by the elevated cellular phenotype (e.g., neutrophilic, eosinophilic, mastocytic). A neutrophils level greater than 20% is indicative of severe asthma; an eosinophils level in the range of 1%-5%, or a mastocytes level in the range of 2%-5% is considered mild. Eosinophils and mastocytes levels of 5% are considered to be moderate. Neutrophils levels between 10-12% are considered to be moderate. A normal horse will have none of these inflammatory cells, or at least no eosinophils and mastocytes, and less than 5% of neutrophils levels. Horses with mastocytic and/or eosinophilic asthma may be referred for additional treatment using a janus kinase inhibitor.

It should be further appreciated that, in other aspects, the disclosed systems and methods can be used to identify other equine conditions including, but not limited to, pneumonia, and other such conditions.

In an embodiment the monitoring system comprises a computer system and software. In some cases, the system is configured for receiving nitric oxide indicia data from the sensor, determining a nitric oxide level from the nitric oxide indicia data, and determining if the nitric oxide level is above a threshold indicative of equine asthma. The software can further be configured for receiving environmental data from at least one of the environmental sensor, and the chemical sensor, analyzing the environmental data, and determining if the environmental data suggests environmental conditions causing equine asthma.

In another embodiment, the software is configured for receiving intradermal (IDT) skin testing data, analyzing the intradermal skin testing data, and determining if the intradermal skin testing data suggests conditions causing equine asthma. In certain embodiments, IDT testing is done manually. The size of the resulting welt is measured and compared to a negative control (saline) and a positive control (histamine). The ratio of those size differences (saline ratio and histamine ratio) are used to determine if there is no response, a mild positive response, a moderate positive response, or a severe positive response. The response can be measured at different times post-injection. In certain embodiments, this data can be input into software, as disclosed herein, for further analysis in conjunction with other data.

The systems disclosed herein can be used in association with methodology for identifying horses likely to have equine asthma, and/or methods for identifying environmental factors that may be the cause of asthma. In certain embodiments, a diagnostic method comprises collecting exhalation from a horse with a collection muzzle, determining the nitric oxide level in the collected exhalation using a sensor, comparing the nitric oxide level in the collected exhalation to a threshold nitric oxide level, and determining if the nitric oxide level is indicative of equine asthma. In certain embodiments the method can further, or alternatively comprise, receiving environmental data from at least one of an environmental sensor, and a chemical sensor and determining if the environmental data suggests environmental conditions are causing equine asthma. In certain embodiments the method can further, or alternatively comprise, receiving intradermal skin testing data and determining if the intradermal skin testing data suggests conditions causing equine asthma.

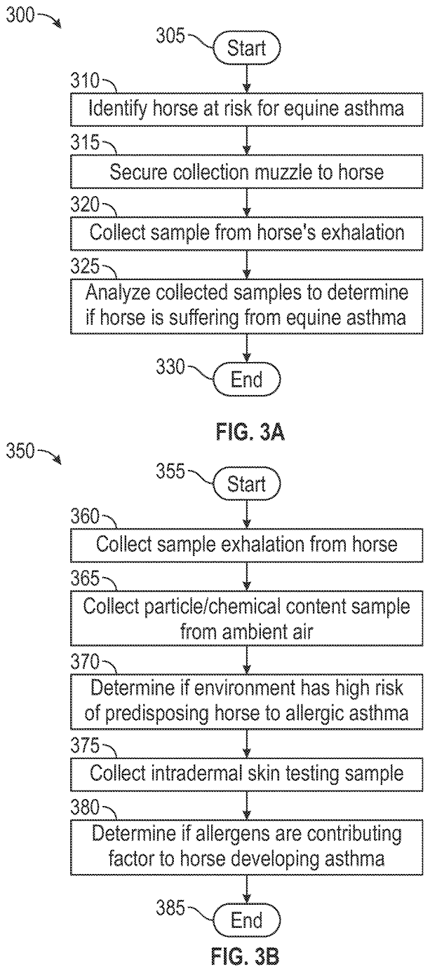

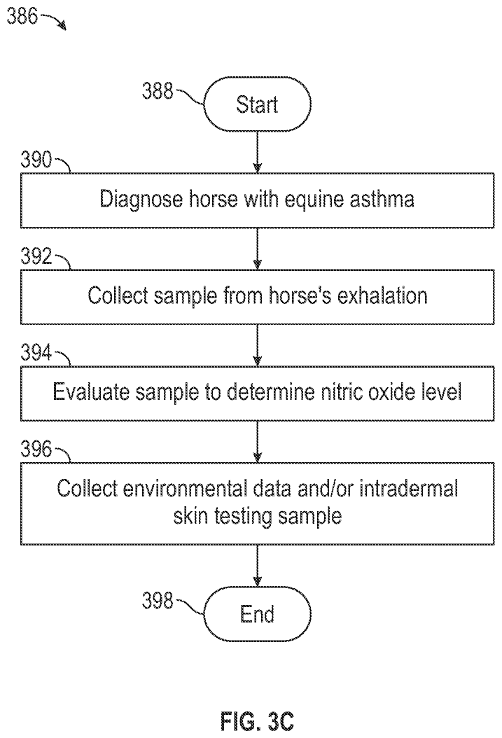

FIG. 3A and FIG. 3B illustrate methods, including method 300 and method 350, for identifying equine asthma or other environmental irritants using the systems disclosed herein. The method 300 begins at step 305.

At step 310 a horse at risk for equine asthma can be identified. This may be based on the animal's athletic performance or according to other environmental risk factors. It may also be a part of standard screening procedures for the animal.

At step 315 a collection muzzle can be secured to the horse. In certain embodiments, this can include securing the muzzle around the horse's nose and mouth so that substantially all, or most of the horse's exhalation is collected in the collection muzzle. At step 320 exhalation from the horse can be collected in the collection muzzle. The sample can be transported to the nitric oxide sensor for testing. These samples can be taken at intervals or continuously.

At step 325 the samples can be analyzed to determine if the animal is suffering from asthma. If the system detects FeNO concentrations ≥20 ppb, there is a high likelihood the horse will be diagnosed with eosinophilic and/or mastocytic asthma.

In some embodiments, the ambient environment can also be sampled in the method 300, at step 325 to identify the particles in the environment surrounding the horse. Similarly, at step 325 the chemical sensor can be used to collect samples of chemicals that may be present in the environment surrounding the horse. Both these samples can be taken at intervals or continuously, and are optional.

The collected samples can be analyzed to determine if the horse is suffering from or otherwise at risk of having equine asthma. Note, the method 300 can similarly be used to identify other maladies in other embodiments. The method ends at 330.

FIG. 3B illustrates another embodiment illustrative of a method 350 for equine allergic airway diagnosis, and associated environmental air and allergen assessment using a monitoring system. The method starts at 355.

At step 360 a muzzle, as disclosed herein, is used to collect gas and/or fluid exhaled by a horse. The exhalation is provided to a sensor used to identify the relative amount of nitric oxide in the horse's exhalation. It should be zero. If it is elevated (i.e., higher than 20 ppb) then there is a high probability the horse has allergic asthma.

At step 365, the environment and air quality can be assessed using sensors such as an environmental sensor and a chemical sensor. If the air quality is poor, meaning that either the particulate load or noxious chemical levels are high, at step 370, it is determined that the environment has a high possibility and probability of predisposing the horse to allergic asthma. It should be appreciated that steps 360 and 365 can be completed in other orders or simultaneously in certain embodiments.

At step 375, the environmental assessment can proceed further with allergy testing via intradermal skin testing of allergens commonly found in the surrounding locale (e.g., Texas, Oklahoma, Nebraska, Colorado, etc.). If the horse has high reactivity to the intradermal skin test at step 375, then at step 380, it is determined that the allergens may be contributing to a high probability of the horse developing asthma. The method ends at step 385.

FIG. 3C illustrates a method 386 for monitoring the efficacy of a therapeutic and/or treatment regimen following diagnosis of equine asthma, in accordance with the disclosed embodiments. For example, in certain embodiments, a horse that is diagnosed with equine asthma, can be monitored to gauge the efficacy of the treatment regime designed to reduce its symptoms. The method starts at 388.

At step 390 a diagnosis of equine asthma can be made. In certain embodiments, this step may be done with, or aided by, the methods illustrated in FIG. 3A and/or FIG. 3B.

After a horse has been identified as having equine asthma, at step 392 exhalation from the asthmatic horse can be periodically collected using the systems and methods disclosed herein. The exhalation can be evaluated at step 394 to identify a level of nitric oxide in the collected exhalation, with a sensor in accordance with the disclosed embodiments. If the nitric oxide level is above a given threshold indicative of equine asthma, it is suggestive that the treatment regime being applied to the horse has not been effective. Conversely, if the nitric oxide level is below a given threshold indicative of equine asthma, it is suggestive that the treatment regime being applied to the horse has been effective. The method 386 can thus be used to monitor the efficacy of a therapeutic and/or treatment regimen for the asthmatic horse.

In certain embodiments, additional data can be collected at step 396. Such data can include environment and air quality assessments using sensors such as an environmental sensors and a chemical sensors disclosed herein. If the air quality is poor, meaning that either the particulate load or noxious chemical levels are high, this may indicate the therapeutic and/or treatment regime should be modified to include steps to reduce the horse's exposure to particulates and/or noxious chemicals. Additional allergy testing via intradermal skin testing of allergens can also be included. If the ITD testing suggests allergens are present, this may indicate the therapeutic and/or treatment regime should be modified to include steps to reduce the horse's exposure to such allergens. The method ends at step 398.

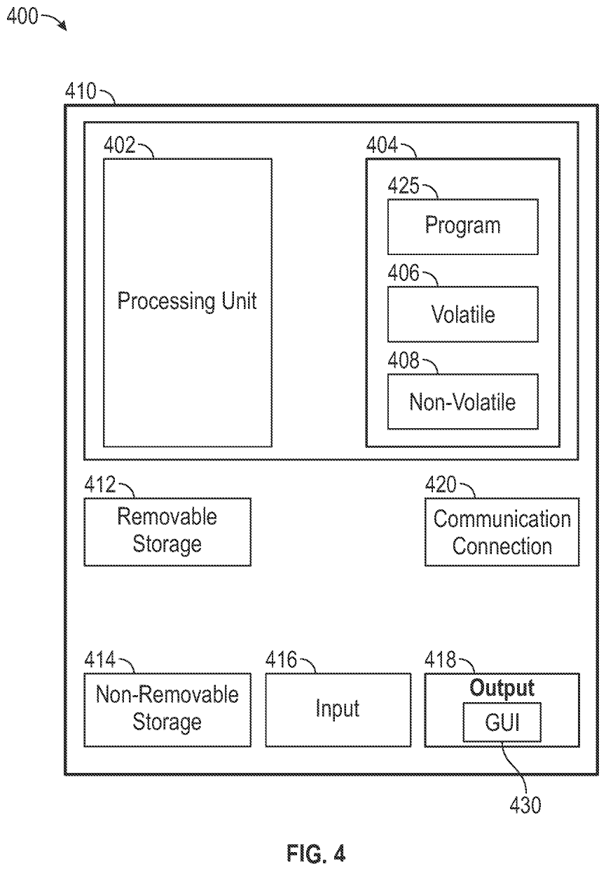

FIGS. 4-6 are provided as exemplary diagrams of data-processing environments in which embodiments may be implemented. It should be appreciated that FIGS. 4-6 are only exemplary and are not intended to assert or imply any limitation with regard to the environments in which aspects or embodiments of the disclosed embodiments may be implemented. Many modifications to the depicted environments may be made without departing from the spirit and scope of the disclosed embodiments.

A block diagram of a computer system 400 that executes programming for implementing parts of the methods and systems disclosed herein is provided in FIG. 4. A computing device in the form of a computer 410 configured to interface with controllers, peripheral devices, and other elements disclosed herein may include one or more processing units 402, memory 404, removable storage 412, and non-removable storage 414. Memory 404 may include volatile memory 406 and non-volatile memory 408. Computer 410 may include or have access to a computing environment that includes a variety of transitory and non-transitory computer-readable media such as volatile memory 406 and non-volatile memory 408, removable storage 412 and non-removable storage 414. Computer storage includes, for example, random access memory (RAM), read only memory (ROM), erasable programmable read-only memory (EPROM) and electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage, or other magnetic storage devices, or any other medium capable of storing computer-readable instructions, as well as data including image data.

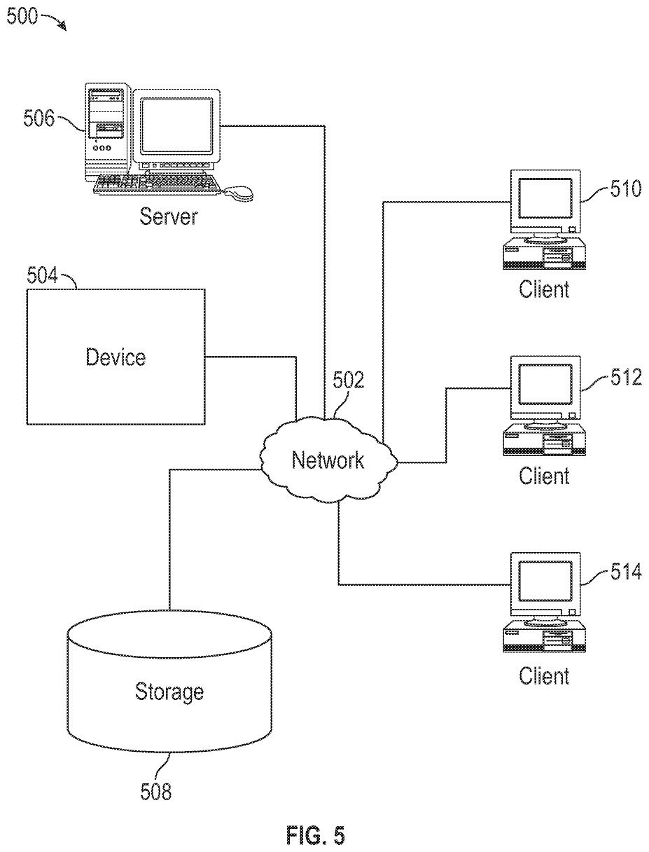

Computer 410 may include, or have access to, a computing environment that includes input 416, output 418, and a communication connection 420. The computer may operate in a networked environment using a communication connection 420 to connect to one or more remote computers, remote sensors and/or controllers, detection devices, hand-held devices, multi-function devices (MFDs), speakers, mobile devices, tablet devices, mobile phones, Smartphone, or other such devices. The remote computer may also include a personal computer (PC), server, router, network PC, RFID enabled device, a peer device or other common network node, or the like. The communication connection may include a Local Area Network (LAN), a Wide Area Network (WAN), Bluetooth connection, or other networks. This functionality is described more fully in the description associated with FIG. 5 below.

Output 418 is most commonly provided as a computer monitor, but may include any output device. Output 418 and/or input 416 may include a data collection apparatus associated with computer system 400. In addition, input 416, which commonly includes a computer keyboard and/or pointing device such as a computer mouse, computer track pad, or the like, allows a user to input instructions to computer system 400. A user interface can be provided using output 418 and input 416. Output 418 may function as a display for displaying data and information for a user, and for interactively displaying a graphical user interface (GUI) 430.

Note that the term “GUI” generally refers to a type of environment that represents programs, files, options, and so forth by means of graphically displayed icons, menus, and dialog boxes on a computer monitor screen. A user can interact with the GUI to select and activate such options by directly touching the screen and/or pointing and clicking with a user input device 416 such as, for example, a pointing device such as a mouse, and/or with a keyboard. A particular item can function in the same manner to the user in all applications because the GUI provides standard software routines (e.g., program module or node 425) to handle these elements and report the user's actions. The GUI can further be used to display the electronic service image frames as discussed below.

Computer-readable instructions, for example, program module or node 425, which can be representative of other modules or nodes described herein, are stored on a computer-readable medium and are executable by the processing unit 402 of computer 410. Program module or node 425 may include a computer application. A hard drive, CD-ROM, RAM, Flash Memory, and a USB drive are just some examples of articles including a computer-readable medium.

FIG. 5 depicts a graphical representation of a network of data-processing systems 500 in which aspects of the present invention may be implemented. Network data-processing system 500 can be a network of computers or other such devices, such as mobile phones, smart phones, sensors, controllers, actuators, speakers, “internet of things” devices, and the like, in which embodiments of the present invention may be implemented. Note that the network data-processing system 500 can be implemented in the context of a software module such as program module or node 425. The network data-processing system 500 includes a network 502 in communication with one or more clients 510, 512, and 514. Network 502 may also be in communication with one or more devices 504, servers 506, and storage 508. Network 502 is a medium that can be used to provide communications links between various devices and computers connected together within a networked data processing system such as computer system 400. Network 502 may include connections such as wired communication links, wireless communication links of various types, and fiber optic cables. Network 502 can communicate with one or more servers 506, one or more external devices such as device 504, and a memory storage unit 508 such as, for example, memory or database It should be understood that device 504 may be embodied as a sensor, detector device, controller, receiver, transmitter, transceiver, transducer, driver, signal generator, testing apparatus, or other such device.

In the depicted example, device 504, server 506, and clients 510, 512, and 514 connect to network 502 along with storage unit 508. Clients 510, 512, and 514 may be, for example, personal computers or network computers, handheld devices, mobile devices, tablet devices, smart phones, personal digital assistants, controllers, recording devices, speakers, MFDs, etc. Computer system 400 depicted in FIG. 4 can be, for example, a client such as client 510 and/or 512 and/or 514.

Computer system 400 can also be implemented as a server such as server 506, depending upon design considerations. In the depicted example, server 506 provides data such as boot files, operating system images, applications, and application updates to clients 510, 512, and/or 514. Clients 510, 512, and 514 and device 504 are clients to server 506 in this example. Network data-processing system 500 may include additional servers, clients, and other devices not shown. Specifically, clients may connect to any member of a network of servers, which provide equivalent content.

In the depicted example, network data-processing system 500 is the Internet, with network 502 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers consisting of thousands of commercial, government, educational, and other computer systems that route data and messages. Of course, network data-processing system 500 may also be implemented as a number of different types of networks such as, for example, an intranet, a local area network (LAN), or a wide area network (WAN). FIGS. 4 and 5 are intended as examples and not as architectural limitations for different embodiments of the present invention.

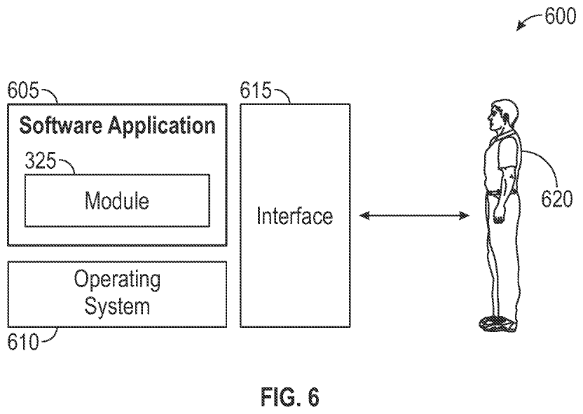

FIG. 6 illustrates a software system 600, which may be employed for directing the operation of the data-processing systems such as computer system 400 depicted in FIG. 4. Software application 605, may be stored in memory 404, on removable storage 412, or on non-removable storage 414 shown in FIG. 4, and generally includes and/or is associated with a kernel or operating system 610 and a shell or interface 615. One or more application programs, such as program module(s) or node(s) 425, may be “loaded” (i.e., transferred from removable storage 412 into the memory 404) for execution by the computer system 400. The computer system 400 can receive user commands and data through user interface 615, which can include input 416 and output 418, accessible by a user 620. These inputs may then be acted upon by the computer system 400 in accordance with instructions from operating system 610 and/or software application 605 and any program module(s) or node(s) 425 thereof.

Generally, program modules (e.g., program modules or nodes 425) can include, but are not limited to, routines, subroutines, software applications, programs, objects, components, data structures, etc., that perform particular tasks or implement particular abstract data types and instructions. Moreover, those skilled in the art will appreciate that elements of the disclosed methods and systems may be practiced with other computer system configurations such as, for example, hand-held devices, mobile phones, smart phones, tablet devices multi-processor systems, microcontrollers, printers, copiers, fax machines, multi-function devices, data networks, microprocessor-based or programmable consumer electronics, networked personal computers, minicomputers, mainframe computers, servers, medical equipment, medical devices, and the like.

Note that the term “module” or “node” as utilized herein may refer to a collection of routines and data structures that perform a particular task or implements a particular abstract data type. Modules may be composed of two parts: an interface, which lists the constants, data types, variables, and routines that can be accessed by other modules or routines; and an implementation, which is typically private (accessible only to that module), and which includes source code that actually implements the routines in the module. The term module may also simply refer to an application such as a computer program designed to assist in the performance of a specific task such as word processing, accounting, inventory management, etc., or a hardware component designed to equivalently assist in the performance of a task.

The interface 615 (e.g., a graphical user interface 430) can serve to display results, whereupon a user 620 may supply additional inputs or terminate a particular session. In some embodiments, operating system 610 and GUI 430 can be implemented in the context of a “windows” system. It can be appreciated, of course, that other types of systems are possible. For example, rather than a traditional “windows” system, other operation systems such as, for example, a real-time operating system (RTOS) more commonly employed in wireless systems may also be employed with respect to operating system 610 and interface 615. The software application 605 can include, for example, program module(s) or node(s) 425, which can include instructions for carrying out steps or logical operations such as those shown and described herein.

The following description is presented with respect to embodiments of the present invention, which can be embodied in the context of, or require the use of, a data-processing system such as computer system 400, in conjunction with program module or node 425, and network data-processing system 500 and network 502 depicted in FIGS. 4-6. The present invention, however, is not limited to any particular application or any particular environment. Instead, those skilled in the art will find that the system and method of the present invention may be advantageously applied to a variety of system and application software including database management systems, word processors, and the like. Moreover, the present invention may be embodied on a variety of different platforms including Windows, Macintosh, UNIX, LINUX, Android, Arduino, LabView and the like. Therefore, the descriptions of the exemplary embodiments, which follow, are for purposes of illustration and not considered a limitation.

Based on the foregoing, it can be appreciated that a number of embodiments, preferred and alternative, are disclosed herein. In an embodiment, a monitoring system comprises a collection muzzle, a sensor in fluidic connection with the collection muzzle, an environmental sensor disposed in an environment proximate to the collection muzzle, and a chemical sensor disposed in the environment proximate to the collection muzzle. In an embodiment, the sensor comprises a nitric oxide monitor. In an embodiment, an environmental sensor comprises a particle monitor. In an embodiment, a particle monitor is configured to detect particles of 10 microns or less. In an embodiment, the chemical sensor further comprises a sensor configured to measure at least one of: ammonia, carbon monoxide, methane, and/or hydrogen sulfide. In an embodiment, the collection muzzle further comprises: a collection chamber and a sealing gasket formed on a rim of the collection chamber. In an embodiment, the collection muzzle further comprises an adaptor port configured to engage attachment tubing connecting the collection chamber to the sensor. In an embodiment, the collection muzzle is configured to cover the nose and mouth of a horse.

In an embodiment, the monitoring system further comprises a computer system comprising at least one processor and a storage device communicatively coupled to the at least one processor, the storage device storing instructions which, when executed by the at least one processor, cause the at least one processor to perform operations comprising: receiving nitric oxide indicia data from the sensor, determining a nitric oxide level from the nitric oxide indicia data, and determining if the nitric oxide level is above a threshold indicative of equine asthma. In an embodiment, the at least one processor performs operations comprising: receiving environmental data from at least one of the environmental sensor, and the chemical sensor, analyzing the environmental data, and determining if the environmental data is indicative of environmental conditions causing equine asthma. In an embodiment, the at least one processor performs operations comprising receiving intradermal skin testing data, analyzing the intradermal skin testing data, and determining if the intradermal skin testing data suggests conditions causing equine asthma.

In an embodiment, a system comprises a collection muzzle configured to fit a horse, a nitric oxide sensor in fluidic connection with the collection muzzle, and attachment tubing connecting the collection muzzle to the nitric oxide sensor. In an embodiment, the system comprises a computer system comprising at least one processor and a storage device communicatively coupled to the at least one processor, the storage device storing instructions which, when executed by the at least one processor, cause the at least one processor to perform operations comprising: receiving nitric oxide indicia data from the nitric oxide sensor. In an embodiment, the collection muzzle further comprises a collection chamber and a sealing gasket formed on a rim of the collection chamber. In an embodiment, the collection muzzle further comprises an adaptor port configured to engage the attachment tubing connecting the collection chamber to the sensor. In an embodiment, the nitric oxide sensor is integrated in the collection chamber.

In an embodiment, a diagnostic method comprises collecting exhalation from a horse with a collection muzzle, determining the nitric oxide level in the collected exhalation using a sensor, and comparing the nitric oxide level in the collected exhalation to a threshold nitric oxide level. In an embodiment, the diagnostic method further comprises determining if the nitric oxide level is indicative of equine asthma. In an embodiment, the method further comprises receiving environmental data from at least one of an environmental sensor and a chemical sensor and determining if the environmental data suggests environmental conditions are causing equine asthma. In an embodiment the method further comprises receiving intradermal skin testing data and determining if the intradermal skin testing data suggests conditions causing equine asthma.

It will be appreciated that variations of the above-disclosed and other features and functions, or alternatives thereof, may be desirably combined into many other different systems or applications. Also, it should be appreciated that various presently unforeseen or unanticipated alternatives, modifications, variations, or improvements therein may be subsequently made by those skilled in the art which are also intended to be encompassed by the following claims.

What is claimed is:

1. A monitoring system comprising:
a collection muzzle;
a sensor in fluidic connection with the collection muzzle;
an environmental sensor disposed in an environment proximate to the collection muzzle; and
a chemical sensor disposed in the environment proximate to the collection muzzle.

2. The monitoring system of claim 1 wherein the sensor comprises:
a nitric oxide monitor.

3. The monitoring system of claim 1 wherein the environmental sensor comprises:
a particle monitor.

4. The monitoring system of claim 3 wherein the particle monitor is configured to detect particles of 10 microns or less.

5. The monitoring system of claim 1 wherein the chemical sensor comprises a sensor configured to measure at least one of:
ammonia;
carbon monoxide;
methane; and/or
hydrogen sulfide.

6. The monitoring system of claim 1 wherein the collection muzzle comprises:
a collection chamber; and
a sealing gasket formed on a rim of the collection chamber.

7. The monitoring system of claim 6 wherein the collection muzzle further comprises:
an adaptor port configured to engage attachment tubing connecting the collection chamber to the sensor.

8. The monitoring system of claim 1 wherein the collection muzzle is configured to cover a nose and mouth of a horse.

9. The monitoring system of claim 1 further comprising:
a computer system comprising at least one processor and a storage device communicatively coupled to the at least one processor, the storage device storing instructions which, when executed by the at least one processor, cause the at least one processor to perform operations comprising:
receiving nitric oxide indicia data from the sensor;
determining a nitric oxide level from the nitric oxide indicia data; and
determining if the nitric oxide level is above a threshold indicative of equine asthma.

10. The monitoring system of claim 9 wherein the at least one processor performs operations comprising:
receiving environmental data from at least one of the environmental sensor, and the chemical sensor;
analyzing the environmental data; and
determining if the environmental data is indicative of environmental conditions causing equine asthma.

11. The monitoring system of claim 9 wherein the at least one processor performs operations comprising:
receiving intradermal skin testing data;
analyzing the intradermal skin testing data; and
determining if the intradermal skin testing data suggests conditions causing equine asthma.

12. A system comprising:
a collection muzzle configured to fit a horse;
a nitric oxide sensor in fluidic connection with the collection muzzle; and
attachment tubing connecting the collection muzzle to the nitric oxide sensor.

13. The system of claim 12 further comprising:
a computer system comprising at least one processor and a storage device communicatively coupled to the at least one processor, the storage device storing instructions which, when executed by the at least one processor, cause the at least one processor to perform operations comprising:
receiving nitric oxide indicia data from the nitric oxide sensor.

14. The system of claim 12 wherein the collection muzzle further comprises:
a collection chamber; and
a sealing gasket formed on a rim of the collection chamber.

15. The system of claim 14 wherein the nitric oxide sensor is integrated in the collection chamber.

16. The system of claim 14 wherein the collection muzzle further comprises:
an adaptor port configured to engage the attachment tubing connecting the collection chamber to the nitric oxide sensor.

17. A diagnostic method comprising:
collecting exhalation from a horse with a collection muzzle;
determining a nitric oxide level in the collected exhalation using a sensor; and
comparing the nitric oxide level in the collected exhalation to a threshold nitric oxide level.

18. The diagnostic method of claim 17 further comprising:
determining if the nitric oxide level is indicative of equine asthma.

19. The diagnostic method of claim 17 further comprising:
receiving environmental data from at least one of an environmental sensor and a chemical sensor; and
determining if the environmental data suggests environmental conditions are causing equine asthma.

20. The diagnostic method of claim 17 further comprising:
receiving intradermal skin testing data; and
determining if the intradermal skin testing data suggests conditions causing equine asthma.

21. A method for monitoring a horse diagnosed with equine asthma comprising:
collecting exhalation from the horse with a collection muzzle;
identifying a level of nitric oxide in the collected exhalation with a sensor; and
determining if the nitric oxide level is above a threshold indicative of equine asthma in order to monitor efficacy of a therapeutic and/or regimen for the horse.

22. The method for monitoring a horse diagnosed with equine asthma of claim 21 further comprising:
receiving environmental data from at least one of an environmental sensor, and a chemical sensor; and
analyzing the environmental data to determine if the therapeutic and/or treatment regimen is being affected by an environment surround the horse.

23. The method for monitoring a horse diagnosed with equine asthma of claim 21 further comprising:
receiving intradermal skin testing data;
analyzing the intradermal skin testing data to identify allergens, in order to determine if the therapeutic and/or treatment regimen is being affected by the allergens.
